(19) United States
(12) Patent Application Publication
(10) Pub. No.: US 2012/0095304 A1
(43) Pub. Date: Apr. 19, 2012

(54) System and Method for Determining a Patient Clinical Status

(75) Inventor: James W. Biondi, North Haven, CT (US)

(73) Assignee: CARDIOPULMONARY CORPORATION, Milford, CT (US)

(21) Appl. No.: 13/282,259

(22) Filed: Oct. 26, 2011

Related U.S. Application Data
(63) Continuation-in-part of application No. 11/640,452, filed on Dec. 15, 2006, now abandoned.
(60) Provisional application No. 60/750,533, filed on Dec. 15, 2005.

(51) Int. Cl. A61B 5/0205; A61B 5/00

(52) U.S. Cl. 600/301

(57) ABSTRACT

A system and method for displaying a patient clinical status. In one embodiment, the system includes a plurality of sensors, each sensor measuring a respective patient parameter; a processor in communication with each of the plurality of sensors, and a display in communication with the processor. The processor receives the patient parameters and generates a patient clinical status in response to the trends of a plurality of patient parameters.

### RELATED APPLICATIONS

This application is a continuation-in-part of U.S. patent application Ser. No. 11/640,452, filed on Dec. 15, 2006, which claims priority to and the benefit of U.S. Provisional Application No. 60/750,533, filed on Dec. 15, 2005, the entire disclosures of each of which are hereby incorporated by reference herein.

### FIELD OF THE INVENTION

The invention relates generally to the field of patient monitoring and more specifically to the field of data analysis used in patient monitoring.

### BACKGROUND OF THE INVENTION

There are a multitude of patient parameters available to the clinician or care provider for monitoring. Many of the parameters comprise real-time physiologic monitoring of the patient. As a result, especially in critical care environments, the amount of data, much of which is time-sensitive, presented to the caregiver is voluminous and as a result the caregiver may not notice trends, or changes in the patient's parameters in a timely, clinically relevant manner. The present invention provides a solution to this problem.

### SUMMARY OF THE INVENTION

The invention relates in one aspect to a system for displaying a patient clinical status. In one embodiment, the system includes a plurality of sensors, each sensor measuring a respective patient clinical or physiologic parameter; a processor in communication with each of the plurality of sensors, and a display in communication with the processor. The processor receives the patient parameters and generates a patient clinical status in response to the plurality of patient parameters. The display displays the patient clinical status generated by the processor.

In one aspect, the invention relates to a system for displaying a patient clinical status. In one embodiment, the system includes a plurality of sensors, each sensor measuring a respective patient parameter; a processor in communication with each of the plurality of sensors, the processor receiving the patient parameters and generating a patient clinical status in response to the trend analysis of plurality of patient parameters; and a display in communication with the processor, the display displaying the patient clinical status. In another embodiment, the patient clinical status is a stability index. In another embodiment, the patient clinical status is a predictive outcome. In another embodiment, the patient clinical status is generated in response to a plurality of trend lines each associated with a patient parameter. In yet another embodiment, the plurality of patient parameters comprise temperature, blood pressure, pulse rate, respiration rate, blood oxygen level, respiration tidal volume, expired respiratory gas, urine output, clinical blood chemistries, or other clinical signs or clinical parameters.

In another aspect, the invention relates to a method for displaying a patient clinical status. In one embodiment, the method includes the steps of measuring a plurality of patient parameters; generating a patient clinical status in response to the trends of a plurality of patient parameters; and displaying the patient clinical status. In another embodiment, the step of generating the patient clinical status further uses a plurality of trending values each associated with a respective patient parameter. In still yet another embodiment, the plurality of patient parameters comprise temperature, blood pressure, pulse rate, respiration rate, blood oxygen level, respiration tidal volume, expired respiratory gas, urine output, clinical blood chemistries, or other clinical signs or physiologic parameters.

Yet another aspect of the invention relates to a system for displaying a patient clinical status. In one embodiment, the system includes a plurality of sensors, each sensor measuring a respective patient parameter; a processor in communication with each of the plurality of sensors, the processor receiving the patient parameters and generating a patient clinical status in response to the trend analysis of plurality of patient parameters; and a display in communication with the processor, the display displaying the patient clinical status. The plurality of patient parameters comprise temperature, blood pressure, pulse rate, respiration rate, blood oxygen level, respiration tidal volume, expired respiratory gas, urine output, clinical blood chemistries, or other clinical signs or physiologic parameters, and the patient clinical status is generated in response to a plurality of trend each associated with a respective plurality of patient parameters.

### BRIEF DESCRIPTION OF THE DRAWINGS

The foregoing and other objects, aspects, features, and advantages of the invention will become more apparent and may be better understood by referring to the following description taken in conjunction with the accompanying drawings, in which:

FIG. 1 is a block diagram of an illustrative embodiment of a system constructed in accordance with the invention; and

FIG. 2 is a graph showing determination of standard deviation of real-time parameters for a smart alarm, in accordance with an illustrative embodiment.

FIG. 3 is a flow chart of an illustrative embodiment of the method performed in accordance with the invention.

FIG. 4 is a graph showing real-time assessment of cardiac/heart failure, in accordance with an illustrative embodiment.

FIG. 5 is a graph showing real-time assessment of sepsis, in accordance with an illustrative embodiment.

FIG. 6 is a graph showing real-time assessment of respiratory failure, in accordance with an illustrative embodiment.

FIG. 7 is a graph showing real-time assessment of brain injury, in accordance with an illustrative embodiment.

FIG. 8 is a graph showing real-time assessment of over sedation, in accordance with an illustrative embodiment.

FIG. 9 is a graph showing real-time assessment of renal failure, in accordance with an illustrative embodiment.

### DETAILED DESCRIPTION

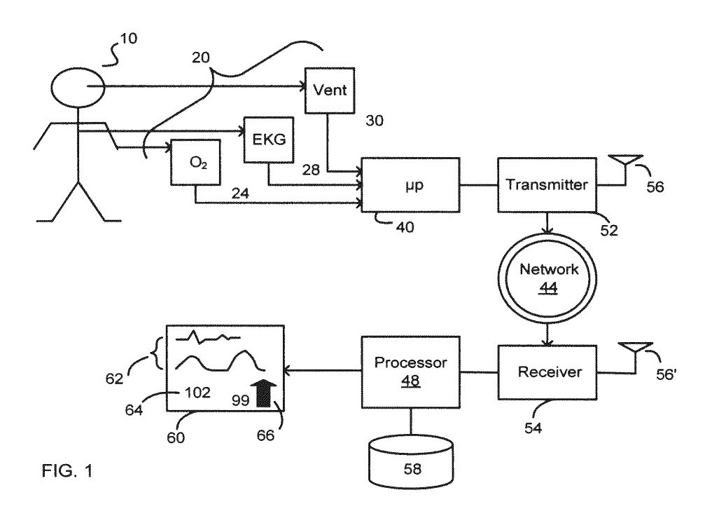

In brief overview and referring to FIG. 1, a patient 10 is monitored by a number of physiologic patient monitors, generally 20. These monitors can include one or more of oxygen sensors 24, carbon dioxide and other metabolic sensors, electrocardiogram (cardiac) 28, hemodynamic (e.g., blood pressure, pulse pressure, blood volume, and blood flow) monitors and ventilation/respiratory monitor 30. Each of these monitors 20 produces one or more signals which are input signals to a processor 40. These signals are processed and either transmitted over a network 44 to a host processor 48 or communicated to a transmitter 52 which broadcasts the signals to a receiver 54 by way of antennae 56, 56′. In one embodiment, the network is hard-wired rather than wireless. The host processor 48 performs calculations on the signals and then transmits the results to a display 60. The display 60 displays the results of the calculations including the original signals 62, a numerical score 64 and a trend indicator 66. In another embodiment, alarms and alerts from the monitors are communicated directly to the display. In other embodiments status change, baseline, clinical trajectory and normal limit indicators are also displayed. In other embodiments, alarms are generated when trends are detected that are detrimental to the patient.

In more detail, the patient 10 has attached a number of sensors each of which is connected to a specific monitor 20. A typical patient 10 might be monitored by an oxygen sensor 24 attached to the patient's finger, airway respiratory gas sensors and detectors, a plurality of electrocardiographic electrodes attached to an EKG monitor 28, hemodynamic sensors (including, but not limited to, blood pressure, pulse pressure, blood flow and blood volume) and a respiratory monitor 30. Each of the monitors 20 produces one or more output signals in response to the input signals provided by the sensors. For example, the oxygen monitor 24 may produce a single value, oxygen concentration in the blood, while the EKG monitor 28 may produce multiple signals, including heart rate and ecg waveforms. Hemodynamic sensors may monitor such parameters as blood pressure, blood flow, blood volume and cardiac output. Body surface sensors and implanted biosensors measure various physiologic functions which are also monitored.

Further, the output signals from the monitors 20 may be substantially the same as received from the sensors or processed. As a result, the signals which are input signals to the processor 40 may be an analog or digital form. If they are in analog form, the input signals are first processed by an analog to digital converter (A/D) before being sent for processing by the processor 48. If the signals are pre-processed by the monitor 20 and have a digital format, input to the processor 40 can be through a serial or parallel digital input device.

The processor 40 then packages the data from the monitors 20 for communication to a transmitter 52. The packaging of the data includes in one embodiment inserting a patient ID number with the data. In addition to packaging the various data for transmission, the processor 40 may also encrypt the data. The transmitter 52, in one embodiment, is a modem to connect the processor 40 to a wired network 44. The network can be a local area or wide area network. In a second embodiment, the transmitter 52 is a WIFI, (or other wireless band) transmitter that transmits the data by way of an antenna 56 over a wireless network. In a third embodiment, the transmitter is a transceiver for transmission of data over a hard-wired network such as RS 232 or ethernet.

The data is received from the network 44 or the WIFI network, through receiver antenna 56′, by a receiver 54 that provides the data to a host processor 48. In another embodiment, the receiver is a transceiver which receives data over a hand-wired network such as an RS 232 or ethernet network. The host processor 48, uses the data for statistical analysis, writes the data to a database 58 and applies rules to the statistically processed data or unprocessed data as described below. The host processor 48 then prepares the data for display on a monitor 60.

The displayed data typically includes the data waveforms 62, but the processed numerical data 64 and calculated values and indicia of status 66. These calculated values include risk indicators and predictive outcome indicators as described below.

In various embodiments, alarms/alerts generated within the monitors are communicated for immediate display. In other embodiments, alarms arising from calculations based on the parameters received from the monitors, such as trend, status change, baseline, clinical trajectory, and normal limit indicators are displayed.

To determine if the patient parameters indicate that the patient is in increasing or decreasing risk, several calculations may be performed. First, a polynomial may be generated which takes into account the parameters of interest, defines their importance by the power of the variable to which the parameter corresponds, and applies a weighting coefficient to each parameter to rank parameters of the same power relative to one another. So for example, an equation in one embodiment is as follows:

Risk index=(A(Hrate−Hrate baseline)^{L}+B(Hirregularity)^{M}+C(O₂−O_{2 ave})^{N}+D(Hrate−Hrate max)^{O}+E(T−Tnormal)^{P}+F(bp−bpbaseline)^{Q}+G(Systolic−Diastolic Pressure)^{R}+H(Pulse Pressure)^{S}+I(Cardiac Output)^{T}+J(Flow/time)^{U}+K(Other Parameter)^{V}

In this equation, deviation from the baseline heart rate (Hrate−Hrate baseline); heartbeat irregularity (Hirregularity); and deviation of blood oxygen concentration from average blood oxygen concentration (O₂−O_{2 min}) are important, but only as a linear function of their deviation. Their relative importance is determined by the values of the coefficients A, B, C through the last parameter coefficient (K). Exponents L, M, N through the last parameter exponent (V) determine the relative importance of the parameter or its deviation from some set valve. For example, deviation from the maximum acceptable heart rate limit (Hrate−Hrate max) and deviation from normal temperature (T−Tnormal) may be more significant and as a result a heart rate greater than the maximum allowable may be raised to the second power and deviation in temperature may be cubed. Thus, in this case deviation from normal temperature and heat rate will have a greater effect on the risk index than a change in O₂ concentration. The coefficients A, B, C are used to weigh the relative importance of the variables which are of the same power. The coefficients may be chosen as a normalizing number make the risk index fall between some values, for example 1 and 100.

In another embodiment, the various parameters are subjected to real-time multivariate analysis as patient data are streamed from medical devices. In various embodiments, after a primary parameter for a clinical outcome crosses a threshold (e.g., body temperature) or fits a predetermined trend (e.g. a sudden decrease in blood pressure), other relevant parameters for that clinical outcome are automatically monitored for changes. If one or more of the other parameters change in a predetermined direction, the patient is considered to be at increased risk for that clinical outcome and an alarm is triggered. In various embodiments, the multivariate analysis includes calculating, for each real-time parameter, standard deviation, variance, and slope analysis/differentiation. In one embodiment, the measured parameters are statistically analyzed in real-time without the need for stored data. The moving statistics, for example a moving average, allow the system to provide a clinician with accurate immediate trends for the measured parameters.

It is important to realize that in trend analysis first, the significance of the magnitude of a change in trend is determined in part by the parameter being measured. For example, a five percent change in heart rate from baseline is not as significant as a five percent change in blood oxygen saturation as measured from baseline. Second, that the significance of the rate of change of the parameter, that is how fast it rises or falls, also depends upon the parameter. Thus, a ten percent decrease in blood pressure over a period of hours may not be significant, but a ten percent decrease in blood pressure over a period of minutes may be significant. Thus, the setting of an alarm based on rate of change of a parameter may vary according to the baseline of the individual patient, the patient population or the experience of the clinician.

Another significant issue is that the various parameters are measured by different medical devices and as such the measurements may be taken at different times, may be taken for different intervals and may actually report different types of parameters (for example average temperature versus instantaneous temperature). All parameter values must be time stamped. Thus when considering the trend of co-dependent parameters, one must consider whether the co-dependent parameter is measured at the same time, whether it is the same type of variable and what the affect of the time delay is on the measurement value of the parameter which caused the program to examine the co-dependency. Thus, depending on the parameter one may have to extrapolate the intermediate values of parameters which are not taken often when comparing them to values of parameters made substantially continuously.

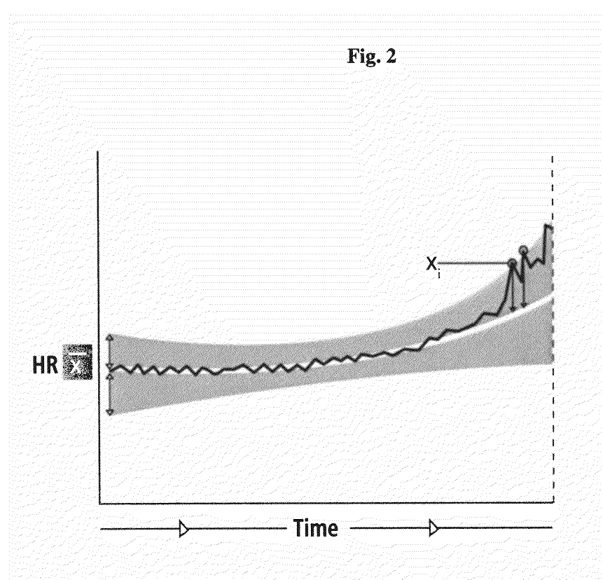

FIG. 2 shows a graph of a standard deviation analysis for an exemplary vital sign parameter (heart rate (HR)). If a patient's vital signs stay steady, the moving average will stay steady and the standard deviation will decrease around the moving average. The moving average can be calculated as follows:

$$\overline{x} = \frac{{\sum }_{i = 1}^{n} {x}_{i}}{n} , \qquad ( 1 )$$

where n=number of data points in sample, xᵢ=measure of parameter at point i, and x̅=mean of all data points (moving average). As heart rate (or any combination of parameters) rises or dips out of the standard deviation bands, an alarm will trigger. Standard deviation (σ), which determines the width above and below the moving average (e.g., the gray band in FIG. 2), can be calculated as follows:

$$σ = \sqrt{\frac{{\sum }_{i = 1}^{n} {( {x}_{i} - \overline{x} )}^{2}}{n}} \qquad ( 2 )$$

Variance measures how far the incoming value is from the moving average of all previous data points. As multiple vital signs deviate from a healthy constant, the increasing variances can trigger an alarm. Variance (σ²) can be calculated as follows:

$${σ}^{2} = {\sum }_{i = 1}^{n} \frac{{( {x}_{i} - \overline{x} )}^{2}}{n} \qquad ( 3 )$$

In addition, as a patient's vitals deviate from a healthy constant, simultaneous slope analyses/differentiation (d or Δ) of multiple parameters can trigger an alarm. The deviation(s) may be used separately to demonstrate a change in clinical trajectory, or in combination as a lumped parameter, as for example, the sum of the absolute values of the changing slopes, to create a clinical status indicator. Slope/differentiation can be calculated for each parameter as follows:

$$\frac{\tilde{d} y}{\tilde{d} x} \sim constant \qquad ( 4 )$$

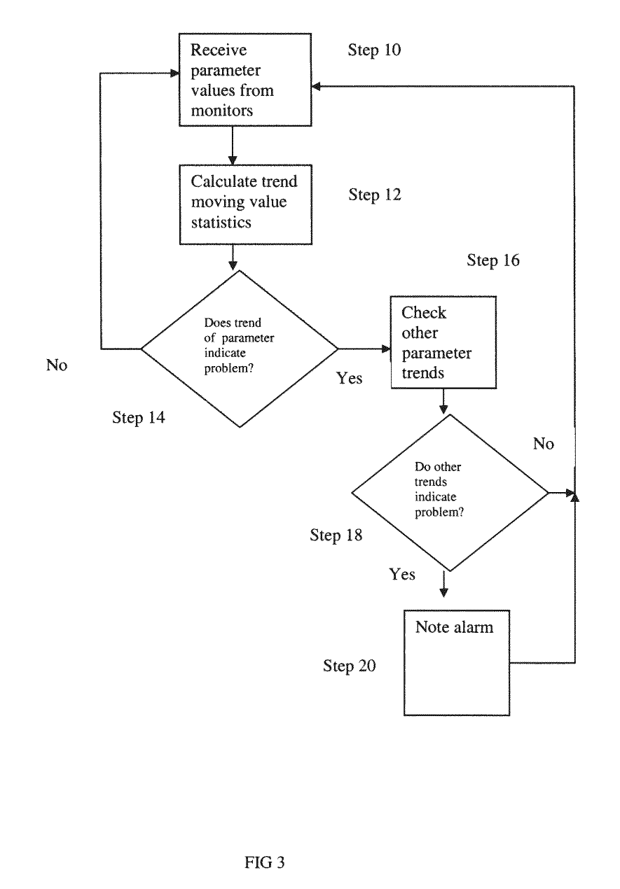

In operation, referring to FIG. 3, the programs of the system receive (step 10) a parameter value from a patient monitor on a given patient. For each parameter moving value statistics are calculated as described above (step 12). If any parameter trend is outside its preset value (step 14), other parameter trends are considered that co-dependent on the parameter that is outside preset value (Step 16). If no parameter trend is outside the preset limit, the program recycles to receive the next parameter values from the patient monitors (Step 10).

If the trends of other parameters that are co-dependent with the parameter that is outside the predetermined limits indicate a problem (Step 18) an alarm is sounded (Step 20) and the programs recycle to receive the next parameter values from the patient monitors (Step 10). If no other co-dependent parameter is found to be outside the predetermined limits, the programs recycle to receive the next parameter values from the patient monitors Step 10).

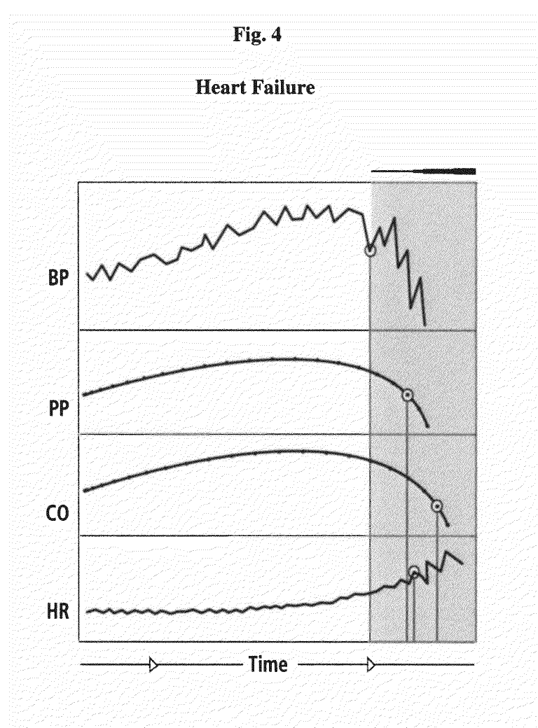

Real-time multivariate analysis is illustrated by the following non-limiting examples. Referring to FIG. 4, cardiac/heart failure can be monitored in real-time by analyzing a cardiac/heart failure profile which can include, for example, blood pressure (BP), pulse pressure (PP), cardiac output (CO), and heart rate (HR). In this example, a decrease in blood pressure of a predetermined amount over a predetermined amount of time (in one embodiment 10 percent over a period of minutes) as measured from a baseline is the primary indicia that the patient is at risk for cardiac failure, and the system then monitors the patient for changes in co-dependent parameters to determine additional trends. Concurrent decreases in pulse pressure and cardiac output combined with an increasing heart rate, as the heart attempts to compensate, indicate the patient is at increasing risk for cardiac failure (depicted by the thickness of a wedge above the graph). A cardiac arrest alarm triggers when a predetermined threshold is crossed.

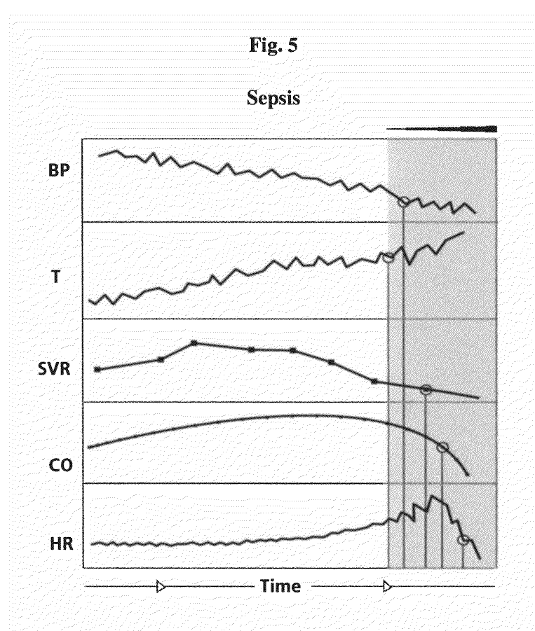

Referring to FIG. 5, sepsis can be monitored in real-time by analyzing a sepsis profile which can include, for example, blood pressure (BP), body temperature (T), systemic vascular resistance (SVR) cardiac output (CO), and heart rate (HR). In this example, an increasing trend in body temperature above a predetermined threshold amount over a baseline value over a predetermined period of time is the primary indicia that the patient is at risk for sepsis, and the system then monitors the patient for trend changes in co-dependent parameters. Concurrent decreases in blood pressure, systematic vascular resistance, and cardiac output, combined with a heart rate that initially increases and then decreases, indicate the patient is at increasing risk for sepsis (again depicted by the thickness of a wedge above the graph). A sepsis alarm triggers when a predetermined threshold is crossed.

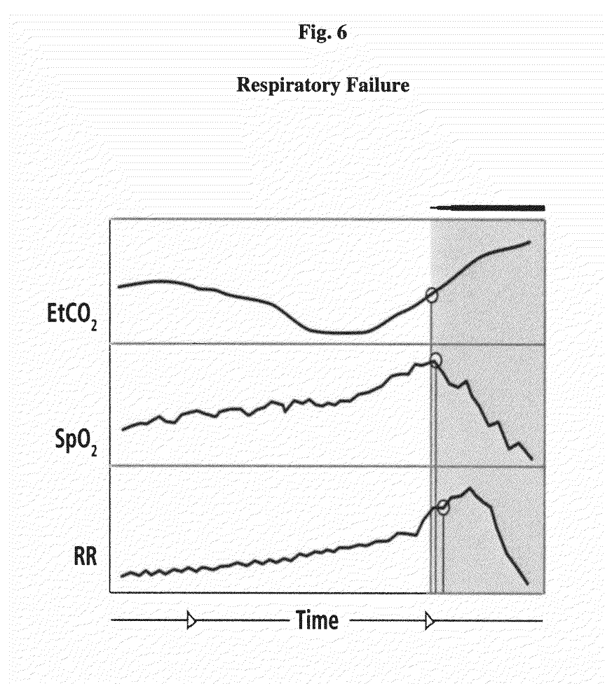

Referring to FIG. 6, respiratory failure can be monitored in real-time by analyzing a respiratory failure profile which can include, for example, end tidal CO₂ (EtCO₂), blood oxygen saturation (SpO₂), and respiratory rate (RR). In this example, a trend of increasing end tidal CO₂ of a predetermined threshold amount over a baseline for a predetermined period of time is the primary indicia that the patient is at risk for respiratory failure, and the system then monitors the patient for changes in co-dependent parameters. Concurrent decreasing trends in blood oxygen saturation combined with a respiratory rate that initially increases and then decreases as the patient tires, indicate the patient is at increasing risk for respiratory failure (depicted by a wedge above the graph). A respiratory failure alarm triggers when a predetermined threshold is crossed.

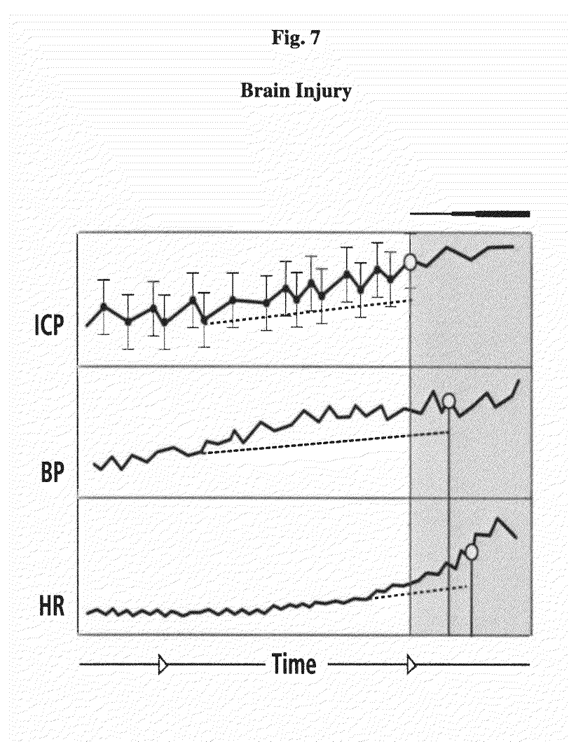

Referring to FIG. 7, brain injury due to swelling can be monitored in real-time by analyzing a brain injury profile which can include, for example, intracranial pressure (ICP), blood pressure (BP), and heart rate (HR). In this example, an increasing trend in intracranial pressure over a predetermined threshold amount above a baseline over a predetermined period of time is the primary indicia that the patient is at risk for brain injury, and the system then monitors the patient for changes in co-dependent parameters. Concurrent increases in blood pressure and heart rate indicate the patient is at increasing risk for brain injury (depicted by a wedge above the graph). A brain injury alarm triggers when a predetermined threshold is crossed.

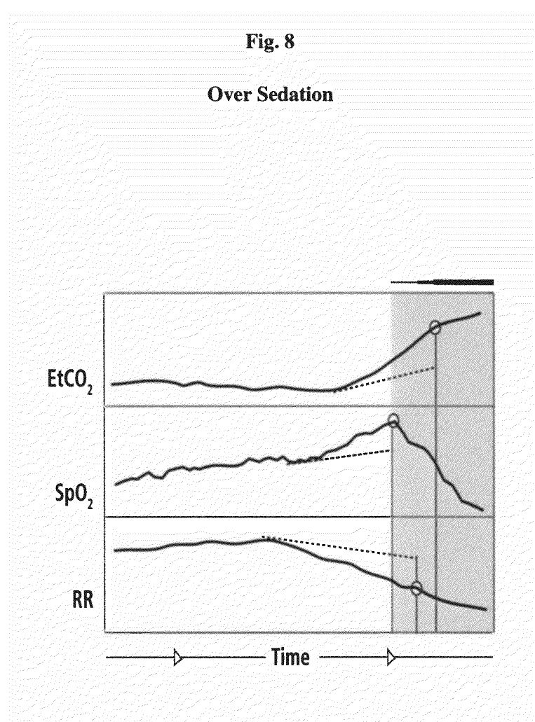

Referring to FIG. 8, patient over sedation can be monitored in real-time by analyzing an over sedation profile which can include, for example, end tidal CO₂ (EtCO₂), blood oxygen saturation (SpO₂), and respiratory rate (RR). Note that these are the same parameters measured for respiratory failure. In this example, a decreasing trend in blood oxygen saturation of a predetermined threshold amount over a baseline value for a predetermined period of time is the primary indicia that the patient is at risk for over sedation, and the system then monitors the patient for changes in co-dependent parameters. Concurrent increases in end tidal CO₂ combined with a declining respiratory rate indicate the patient is at increasing risk for over sedation (depicted by a wedge above the graph). Also note that in discriminating between respiratory failure and over sedation the trend line in respiratory rate discriminates between the two disease states. In over sedation, the respiratory rate never increases due to interference with neural control. In respiratory failure, the rate initially increases until the patient becomes too exhausted to breathe. An over sedation alarm triggers when a predetermined threshold is crossed.

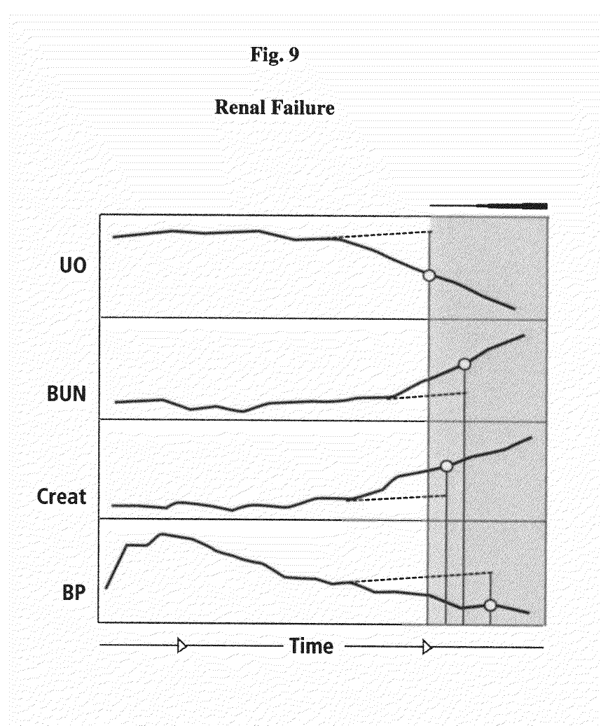

Referring to FIG. 9, renal failure can be monitored in real-time by analyzing a renal failure profile which can include, for example, urinary output (UO), blood urea nitrogen (BUN), creatinine (Creat), and blood pressure (BP). In this example, a decrease in urinary output above a predetermined threshold amount over a baseline value over a predetermined period of time is the primary indicia that the patient is at risk for renal failure, and the system then monitors the patient for changes in co-dependent parameters. Note that in this example the analysis can only be as immediate as the blood chemistry measurements will permit. Concurrent increases in blood urea nitrogen and creatinine combined with decreasing blood pressure, indicate the patient is at increasing risk for renal failure (depicted by a wedge above the graph). A renal failure alarm triggers when a predetermined threshold is crossed.

In one embodiment, the patient parameters as measured by the medical devices is transferred to the system as described above. Part of this data flow is sent to a statistics program whose output is the input to a rule-based system. The rule based system has access to all the parameters but relies on its rule base to make decisions based on the parameters which fall outside expected values.

The methods and systems described herein can be performed in software on general purpose computers, servers, or other processors, with appropriate magnetic, optical or other storage that is part of the computer or server or connected thereto, such as with a bus. The processes can also be carried out in whole or in part in a combination of hardware and software, such as with application specific integrated circuits. The software can be stored in one or more computers, servers, or other appropriate devices, and can also be kept on a removable storage media, such as a magnetic or optical disks. Furthermore, the methods described herein can be implemented using as an SDK, an API, as middleware, and combinations thereof.

The foregoing description has been limited to a few specific embodiments of the invention. It will be apparent, however, that variations and modifications can be made to the invention, with the attainment of some or all of the advantages of the invention. It is therefore the intent of the inventors to be limited only by the scope of the appended claims.

What is claimed is:

1. A system for displaying a patient clinical status comprising:
a plurality of sensors, each sensor measuring a respective patient parameter;
a processor in communication with each of the plurality of sensors, the processor receiving the patient parameters and generating a patient clinical status in response to the trend analysis of plurality of patient parameters; and
a display in communication with the processor, the display displaying the patient clinical status.

2. The system of claim 1 wherein the patient clinical status is a predictive outcome.

3. The system of claim 1 wherein the patient clinical status is generated in response to a plurality of trend lines each associated with a patient parameter.

4. The system of claim 1 wherein the plurality of patient parameters comprise temperature, blood pressure, pulse rate, respiration rate, blood oxygen level, respiration tidal volume, expired respiratory gas, urine output, clinical blood chemistries, or other clinical signs or physiologic parameters.

5. The system of claim 1 wherein the patient clinical status indicator is calculated from a plurality of parameters in a moving value analysis.

6. A method for displaying a patient clinical status, the method comprising the steps of:
measuring a plurality of patient parameters;
generating a patient clinical status in response to the trends of a plurality of patient parameters; and
displaying the patient clinical status.

7. The method of claim 6 wherein the step of generating the patient clinical status further uses a plurality of trending values each associated with a respective patient parameter.

8. The method of claim 6 wherein the plurality of patient parameters comprise temperature, blood pressure, pulse rate, respiration rate, blood oxygen level, respiration tidal volume, expired respiratory gas, urine output, clinical blood chemistries, or other clinical signs or physiologic parameters.

9. A system for displaying a patient clinical status comprising:
a plurality of sensors, each sensor measuring a respective patient parameter;
a processor in communication with each of the plurality of sensors, the processor receiving the patient parameters and generating a patient clinical status in response to the trend analysis of plurality of patient parameters; and
a display in communication with the processor, the display displaying the patient clinical status,
wherein the plurality of patient parameters comprise temperature, blood pressure, pulse rate, respiration rate, blood oxygen level, respiration tidal volume, expired respiratory gas, urine output, clinical blood chemistries, or other clinical signs or physiologic parameters, and
wherein the patient clinical status is generated in response to a plurality of trend lines each associated with a respective plurality of patient parameters.
